(19) United States
(12) Patent Application Publication
(10) Pub. No.: US 2007/0043102 A1
(43) Pub. Date: Feb. 22, 2007

(54) Oral suspension of tegaserod

(76) Inventors: Marie-Noelle Bizot, BOIS D'ARCY (FR); Mark Thomas Iwicki, Long Valley, NJ (US); Christian Vitzling, Paris (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/551,218

(22) PCT Filed: Mar. 30, 2004
(86) PCT No.: PCT/EP04/03368

§ 371 Date: Jul. 24, 2006

(30) Foreign Application Priority Data
Mar. 31, 2003 (GB) 0307440.8

(51) Int. Cl. A61K 31/405; A61K 47/00

(52) U.S. Cl. 514/419; 424/439

(57) ABSTRACT

This invention relates to an oral suspension of tegaserod or pharmaceutically acceptable salts thereof. In particular, it relates to an oral suspension comprising a power comprising an effective amount otegaserod or a pharmaceutically acceptable salt thereof in a beverage, e.g. apple juice.

This invention relates to an oral suspension of tegaserod or pharmaceutically acceptable salts thereof. In particular, it relates to an oral suspension comprising a powder comprising an effective amount of tegaserod or a pharmaceutically acceptable salt thereof in a beverage, e.g. apple juice.

Tegaserod (3-(5-methoxy-1H-indol-3-yl-methylene)-N-pentylcarbazimidamide) and pharmaceutically acceptable salts thereof (also “the Active Agents of the invention”), e.g. hydrogen maleate or hydrochloride, and their manufacture are known e.g. from EP 505322 (herein incorporated by reference) and under the trademarks ZELMAC® and ZELNORM®. The Active Agents of the invention are serotonergic active agents acting on the gastro-intestinal system as partial agonists of the 5-HT₄ receptor and are useful for the prevention and treatment of gastrointestinal motility disorders, e.g. Irritable Bowel Syndrome (IBS), Gastro-Esophageal Reflux Disease (GERD), Functional Dyspepsia (FD), Post Operative leus (POI), Diabetic gastroporesis and chronic constipation. Published PCT Application WO 00/10526 describes solid oral pharmaceutical compositions of e.g. tegaserod with its use in gastrointestinal motility disorders including the solid tablet formulation of tegaserod which is at time of filing this application the currently being marketed tablet form in several countries, e.g. the U.S. and Australia (herein after referred to as “the marketed tablet of tegaserod” or “the currently marketed tablet of tegaserod”).

Despite the merits of the above-mentioned compositions, there remains the problem for patient who have difficulties to take their tegaserod formulation, e.g. have difficulties to swallow the solid dosage form. For example, elderly or senile patients, mentally handicapped patients, severely injured patients, patients after surgery, e.g. suffering from POI may require special administration of the tegaserod drug, e.g. in the form of beverage. Thus there is a marketing need to have a liquid formulation for tegaserod with acceptable criteria of dissolution, stability and homogeneity. At the moment, tegaserod is not commercially available as a liquid formulation at all but only as a solid tablet. Furthermore, tegaserod is only commercially available as a 6 mg and in a few countries as a 2 mg tablet. Both tablet formulations do not have a partition line. Thus there is no straight forward possibility for a doctor to administer tegaserod in a particular dosage other than 6 or 2 mg. As the herein disclosed oral suspensions of tegaserod are homogenous, the invention allows partitioning of the dose with ease. Moreover, the mixture of crushed tablets of tegaserod with particular aqueous solutions has a sufficient stability profile.

The present invention relates in a first aspect to an oral suspension comprising a mixture in divided form comprising an effective amount of tegaserod or a pharmaceutically acceptable salt thereof, and a beverage.

More preferably the present invention relates in a first aspect to an oral suspension comprising a mixture in a divided form comprising an effective amount of tegaserod or a pharmaceutically acceptable salt thereof, and a beverage; said suspension having a dissolution characteristic for tegaserod in water of more than about 80% after 30 minutes, preferably more than about 90% after 30 minutes, more preferably more than about 80% after 5 minutes, and more preferably more than about 90% after 5 minutes.

The oral suspension has preferably a pH above about 2, more preferably above about 3. The beverage may be added to the powder at a ratio of e.g. 6 mg tegaserod and e.g. about. 10 to about 100 ml, more preferably to about 50 ml.

The preferred pharmaceutically acceptable salt of tegaserod is the hydrogen maleate salt.

For the purpose of this invention, the beverage is defined as any liquid that is fit for drinking (The Roget's II: The New Thesaurus, Third Edition, 1995). It may be a drink of any type that can be purchased e.g. in a supermarket or obtained from the tap. The beverage can be e.g. milk, water, apple juice or orange juice.

For the purpose of this invention, a “mixture in divided form” may be a powder, a granulate, a grind, a pulver or particles.

In a preferred embodiment, the present invention relates to an oral suspension as described above; whereas said mixture in a divided form is a crushed tablet. The crushed tablet may be obtained by crushing a tablet of tegaserod, preferably the currently marketed tablet of tegaserod, more preferably one tablet of the currently marketed tablet of tegaserod, e.g. as described below. The crushed tablet should be understood as a part or parts of a tablet or one or more tablets, preferably one tablet.

In a further preferred embodiment, the present invention relates to an oral suspension as described above; whereas the beverage has a task-masking effect, able to overcome the bitter taste of tegaserod, e.g. is apple juice.

In a further aspect the present invention relates to a process to make the mixture in divided form, e.g. powder as described above comprising an effective amount of tegaserod or a pharmaceutically acceptable salt thereof. The process comprises the steps of packing a tablet comprising an effective amount of tegaserod or a pharmaceutically acceptable salt thereof in a piece of aluminum foil and crushing said tablet into a mixture in divided form, e.g. powder.

A preferred preparation of the oral suspensions comprises the following steps:
take out one 6 mg marketed tablet of tegaserod from the blister
cut a piece of aluminum foil (food quality) of approx 10×10 cm
place the Tablet on the center of the aluminum foil piece
bend double the aluminum foil piece and fold back the 3 open sides in order to get a closed pouch with the tablet inside
place the aluminum pouch on a flat and hard surface (e.g. on a table)
take a tea-spoon and crush the tablet firmly with the back of the spoon through the aluminum foil until the tablet breaks into a mixture in divided form
open carefully the aluminum pouch, in order to avoid any loss of mixture in divided form, and pour the mixture in divided form into the beverage, e.g. 50 ml apple juice
carefully mix with a tea spoon (or a spatula) during one minute.

In a further aspect, the present invention relates to an oral suspension as described above, whereas said mixture having the dissolution characteristics in water of

| Time (minutes) | % of theoretical content dissolved (mean) |
|---|---|
| 5 | >80 |
| 15 | >85 |
| 30 | >90. |

The oral suspensions of the present invention are useful in the known indications of the particular active agent incorporated therein. For example, said oral suspensions are useful in the prevention and treatment of gastro-intestinal disorders and anal continence dysfunctions, e.g. in Irritable Bowel Syndrome (IBS), Functional Dyspepsia (FD) and gastroesophageal reflux disease (GERD).

Tegaserod is known. Its manufacture and therapeutic use in the prevention and treatment of gastrointestinal disorders and anal continence dysfunctions are described in e.g. EP 505322 which is incorporated herein by reference. Commercially available dosage forms are provided for oral administration, for example tablets comprising 2 and 6 mg of active ingredient. Those dosage forms are known by the trademark ®Zelmac or ®Zelnorm (Novartis) and have been introduced in a large number of countries, such as the U.S., Australia, South Korea and Switzerland.

The exact amounts of active agent, tegaserod, and of the oral suspension to be administered depend on a number of factors, e.g. the condition to be treated, the desired duration of treatment and the rate of release of active agent.

For example, the amount of tegaserod required and the release rate thereof may be determined on the basis of conventional in vitro and in vivo techniques, determining how long the tegaserod blood plasma level remains at an acceptable level for a therapeutic effect. Examples of doses provided in a solid formulation to human patient (=effective amount of tegaserod), e.g. a tablet, are 0.5 to 12 mg BID (=twice a day) of tegaserod, for IBS and 0.5 to 12 mg BID and 0.5 to 6 mg TID (=three times a day) of tegaserod for FD and 0.2 to 12 mg BID of tegaserod for GERD, irrespective of the body weight.

The following examples illustrate the invention.

### EXAMPLES

The following beverages and tablets are used for the Examples:

| | |
|---|---|
| Water | (drinking water from tap) |
| Apple juice | (Auchan) |
| Orange juice | (Tropicana, pure premium) |
| Milk | (skimmed milk, Auchan) |

Composition of a 6 mg tegaserod tablet (according to Example 3 and 4 of WO 01/0526):

| | | |
|---|---|---|
| Tegaserod maleate | 8.31 | mg (6 mg base) |
| Polyplasdone XL USP/NF | 50.00 | mg |
| Glyceryl monostereate USP/NF | 12.50 | mg |
| Poloxalkol | 2.50 | mg |
| Lactose 200 mesh | 37.94 | mg |
| HPMC 3 cPs | 6.25 | mg |
| Polyethyleneglycol 4000 | 7.50 | mg |
| Water adsorbed | 3.00 | mg |
| Total | 128 | mg |

Composition of a 2 mg tegaserod tablet (according to Example 1 and 2 of WO 01/0526):

| | | |
|---|---|---|
| Tegaserod maleate | 2.77 | mg (2 mg base) |
| Polyplasdone XL USP/NF | 36.00 | mg |
| Glyceryl monostereate USP/NF | 9.00 | mg |
| Poloxalkol | 1.80 | mg |
| Lactose 200 mesh | 30.53 | mg |
| HPMC 3 cPs | 4.50 | mg |
| Polyethyleneglycol 4000 | 5.40 | mg |
| Water adsorbed | 2.00 | mg |
| Total | 92 | mg |

### Example 1

### Stability Study of the Oral Suspensions of the Present Invention

The stability study is performed for maximum 3 days at the storage conditions of room temperature (about 20-25° C.) and/or 5° C. ( fridge). The start point (TO) is performed about 1 minute after preparation. Furthermore, a “24 hours” and “3 days” time point at room temperature and 5° C. is performed.

Analytical methods: For each time-point tested, the degradation products of Tegaserod by HPLC are evaluated. Analytical methods are based on Test Methods described below.

Procedure for assay determination: For each time-point, one tablet is crushed according to the above procedure. The crushed tablet is transferred quantitatively into a 100 ml amber glass volumetric flask. 50 ml of the beverage is carefully added and the suspension is shaken vigorously by hand a few time and allowed to stand for the storage time tested. The suspension is re-homogenized, by shaking by hand. For extraction, the flask is filled to the mark with methanol (about 50 ml) then shaken for 30 minutes under magnetic stirring, sonicated for 10 minutes and shaken for 30 minutes under magnetic stirring. An aliquot of the methanol solution is centrifuged. The clear supernatant of the above solution is used as the test solution and is analyzed by HPLC (Isocratic HPLC on reversed phase RP-8 with UV detection). The same procedure is carried out once for each beverage but without tablet powder and the resulting chromatograms are compared for new appearance of any peak.

Procedure for determination of degradation products: In order to enable to reach a sufficiently low limit to quantify the degradation products, the number of tablets to be crushed is increased. For each time-point, 6 tablets for 2 mg dosage strength (or 5 tablets for 6 mg dosage strength) are crushed according to the above procedure. The crushed tablet is transferred quantitatively into a 20 ml for 2 mg dosage strength (or 50 ml amber glass volumetric flask for 6 mg dosage strength). 10 ml for 2 mg dosage strength (or 25 ml for 6 mg dosage strength) of the beverage are added carefully. The suspension is shaken vigorously by hand a few time and is allowed to stand for the storage time tested. The suspension is re-homogenized by shaking by hand. For extraction, the flask is filled to the mark with acetonitrile and is shaken for 30 minutes under magnetic stirring, sonicated for 10 minutes and shaken for 30 minutes under magnetic stirring. An aliquot of the homogenized solution is centrifuged.

The clear supernatant of the above solution is used as the test solution in order to quantify using a gradient HPLC on reversed phase RP-8 with UV detection. The same procedure is carried out once for each beverage but without crushed tablet and the resulting chromatograms are compared and analyzed according to the scheme below.

Acceptance criteria: Tegaserod is assumed stable in the tested media if the following acceptance criteria are met for the time-points studied.

| Table of Acceptance Criteria | | Values of acceptance |
|---|---|---|
| Assay | start | 90.0-110.0% |
| | after storage | Not more than 3% less than the |
| | | start value* |
| Degradation | For all time-points | ≦0.5%** |
| product | | |
| 5-methoxy-1H- | | |
| indole-3- | | |
| carbaldehyde | | |
| Degradation | For all time-points | Each individual ≦0.2%** |
| products which | | All together: ≦1.0%** |
| are not known | | |

*2% (usual difference allowed between 2 analysis of the same sample generated in 2 different laboratories) + 1% (sum of degradation product allowed)
**Current specifications (stability) for Degradations Products for Zelmac tablets

Tegaserod is stable in apple juice, orange juice and water for at least 1 hour at room temperature and for up to 3 days in the fridge (for 6 mg dosage strength) or for at least 24 hours in the fridge (for 2 mg dosage strength).

### Example 2

### Completeness of Administration and Homogeneity of the Content

The following beverages are used for the study:

| | |
|---|---|
| Tap water | (drinking water from tap) |
| Apple juice | (Auchan) |
| Orange juice | (Tropicana, pure premium) |
| Milk | (half skimmed, Auchan) |

When taking the medication with a beverage, the procedure followed by the patient comprises crushing of the tablets, mixing the crushed tablet with the beverage in a glass, and drinking the content of the glass. It is important to verify whether under those conditions, the complete dose is taken by the patient. Consequently the test described below as “completeness of administration” is performed (this is described by the assay method below). In addition, the homogeneity of the content of tegaserod in all solutions is tested in order to determine if the mixture could be apportioned to administer lower doses.

Analytical procedure: The test is performed using HPLC as above (reversed phase RP-8 with UV detection):

The crushed tablet resulting from the crushing of one tablet is transferred quantitatively into a glass containing about 50 ml of the tested beverage. The suspension is stirred using a tea spoon.
For completeness of administration: the content of the glass is transferred quantitatively to a 100 ml amber glass volumetric flask (this simulate the drinking of the beverage by the patient: the glass was NOT rinsed) which is immediately filled with to the 100 ml mark methanol.
For homogeneity of content: 15 ml of the solution in the glass (exactly 30 % of the totality of the solution) is withdrawn and transferred quantitatively to a 100 ml amber glass volumetric flask and immediately fill to the 100ml mark with methanol.

For extraction, the flask is shaken for 30 minutes under magnetic stirring, sonicated for 10 minutes and shaken for 30 minutes under magnetic stirring. An aliquot of the homogenized solution is centrifuged.

The clear supernatant of the above solution is used as the test solution in order to quantify using a iSocratic HPLC on reversed phase RP-8 with UV detection. The same procedure is carried out once for each beverage but without crushed tablet and the resulting chromatograms are compared and analyzed according to the scheme below.

Acceptance Criteria

| Table of Acceptance Criteria | | Values of acceptance |
|---|---|---|
| Assay | Whole amount of the | 90.0-110.0% of theoretical content |
| | patient sample | |
| | analyzed | |
| | Fraction of the patient | For information only. |
| | sample analyzed | |

The homogeneity of the mixtures with apple juice, water and orange juice is judged adequate and would allow fractionating of the dose. No loss of drug product during administration according to the recommended procedure for the suspensions with apple juice, water and orange juice is observed. Suspensions with milk are considered inhomogeneous as only about 70% of the dose would be taken by the patient (due to probably adsorption to the glass). In addition, the drug substance is not homogeneously distributed in the oral suspension.

### Example 3

### In Vitro Dissolution Tests

The following beverages and food are used for the study:

| | |
|---|---|
| Tap water | (drinking water from tap) |
| Apple juice | (Auchan) |
| Orange juice | (Tropicana, pure premium) |

In vitro dissolution tests are performed according to the following conditions : dissolution medium =water (500 ml), USP apparatus 2 at 50 rpm (apparatus described in the United States Pharmacopeia, type 2 =rotating paddle apparatus; 50 rpm rotating speed of the paddle). All tests are performed on 6 units. Quantitative determination of dissolved tegaserod in medium is done with Isocratic HPLC on reversed phase RP-8 with UV detection.

The test is done with:
the crushed tablets
crushed tablets dispersed in 50 ml of orange juice
crushed tablets dispersed in 50 ml of apple juice

Tablets:

For comparison, the dissolution profiles of the same tablets batch are used.

Crushed Tablets in Water:

Dissolution is performed for 3 units by addition of the crushed tablet into the 500 ml of dissolution medium. Since wettability problem is expected with the crushed tablet, for the 3 other units, the crushed tablet is put at the bottom of the dissolution vessel prior to addition of the dissolution medium. No significant difference is observed for results of both operational procedure, consequently averaged results on 6 units are reported.

Crushed Tablets in Juices:

The powder resulting from the crushed tablet according to the procedure above is added to 50 ml of juice in a glass and homogenized using a spatula. The mixture is added to 450 ml of dissolution medium without rinsing the glass. As a result, the volume of the dissolution medium is 500 ml. No significant difference is observed between the dissolution profile of the crushed tablets and of the tablets. The only difference observed is the higher amount already dissolved at 5 min for the crushed tablet which is due to the fact that no disintegration of the tablet is needed prior to dissolution. After 15 min dissolution profiles are comparable and a complete dissolution is observed for all 6 units, i.e. more than 85 % dissolved. Dissolution profile of crushed tablets are thus the same as in the specifications for Zelmac tablets. The same conclusion applies to the apple juice. No significant difference is observed between the dissolution profile of the crushed tablets mixed with apple juice and the one of the tablets in water. The dissolution profile of the orange juice is less favorable, i.e. around 58% after 60 minutes.

### Example 4

### Masking of the Bitter Taste of Tegaserod

The palatability of the resulting suspensions is determined by the subjective impression of one test person. Thus, it is found by this evaluation that the bitter taste of tegaserod is masked in apple juice and in orange juice but not in water.

1.) An oral suspension comprising a mixture in divided form comprising an effective amount of tegaserod or a pharmaceutically acceptable salt thereof and a beverage.

2.) The oral suspension of claim 1; having a dissolution characteristic in water of more than about 80% after 30 minutes.

3.) The oral suspension of claim 1; whereas the dissolution characteristic in water is more than about 90% after 30 minutes.

4.) The oral suspension of claim 1; whereas the dissolution characteristic in water is more than about 80% after 5 minutes.

5.) The oral suspension of claim 1; wherein said mixture in divided form is a crushed tablet comprising an effective amount of tegaserod or a pharmaceutically acceptable salt thereof.

6.) The oral suspension of claim 5; wherein the crushed tablet is one crushed tablet comprising an effective amount of tegaserod.

7.) The oral suspension of claim 1; wherein the crushed tablet is one crushed tablet comprising an effective amount of tegaserod maleate.

8.) The oral suspension of claim 1; wherein the crushed tablet is one crushed tablet comprising 6 mg tegaserod.

9.) The oral suspension of claim 1; wherein the crushed tablet is one crushed tablet comprising 2 mg tegaserod.

10.) The oral suspension of claim 1 whereas the beverage is able to mask the bitter taste of tegaserod.

11.) The oral suspension of claim 1; wherein the beverage is apple juice.

12.) The oral suspension of claim 1; wherein the beverage is water.

13.) The oral suspension of claim 1; comprising 10 to 100 ml of the beverage.

14.) The oral suspension of claim 13; wherein the beverage is apple juice.

15.) A process to prepare an oral suspension, comprising packing a tablet comprising an effective amount of tegaserod or a pharmaceutically acceptable salt thereof in a piece of aluminum foil and crushing said tablet into a mixture in divided form.
